(19) United States
(12) Patent Application Publication
(10) Pub. No.: US 2012/0209301 A1
(43) Pub. Date: Aug. 16, 2012

(54) IMPLANT FOR HERNIA REPAIR

(75) Inventors: Stephen Graham Bell, Roma (IT); Giuseppe Amato, Palermo (IT)

(73) Assignee: INSIGHTRA MEDICAL, INC.

(21) Appl. No.: 13/443,266

(22) Filed: Apr. 10, 2012

Related U.S. Application Data
(63) Continuation-in-part of application No. 12/183,930, filed on Jul. 31, 2008.
(60) Provisional application No. 61/030,439, filed on Feb. 21, 2008.

(51) Int. Cl. A61B 17/03

(52) U.S. Cl. 606/151

(57) ABSTRACT

A hernia repair implant includes a first layer made of mesh for facing a body structure having a hernia defect to cover the defect while promoting tissue growth into the first layer from the body structure. The implant also includes a second layer opposed to the first layer and that extends radially beyond the first layer. The second layer is made of anti-adhesion material to prevent tissue growth into the second layer from body structures contacting it. The implant also includes a first elongated centering strap connected to the first layer at a first radial location that extends radially beyond a periphery of the first layer, and a first elongated fixation strap connected to the first layer at a second radial location that is more distanced from a center of the first layer than the first radial location and that extends radially beyond a periphery of the first layer.

Priority is claimed to U.S. provisional patent application 61/598,254, filed Feb. 13, 2012, and to U.S. patent application Ser. No. 12/183,930, filed Jul. 31, 2008 and published as USPP 2009/0216253, of which this application is a continuation in part. Both of the above applications are incorporated herein in their entirety.

### FIELD OF THE APPLICATION

The present invention relates generally to the repair of defects in muscular structures, and more particularly to implants to address ventral wall hernias, inguinal hernias, and methods for advancing the implants into a patient less invasively.

### BACKGROUND OF THE INVENTION

The above-referenced patent publication discloses a surgical implant with both a tension free and fixation free implant mesh having multiple straps extending radially outward from the implant mesh. The straps are pulled through the ventral (abdominal) wall musculature to fix the implant mesh to the ventral wall such that when implanted the implant mesh is in a slackened condition relative to the ventral wall. The implant mesh is sized to be substantially larger than the hernia. To permit tissue ingrowth from the ventral wall into the mesh while preventing undesirable ingrowth of structures in the peritoneal space such as the bowel into the mesh, the mesh is backed with an anti-adhesion layer or substance. A non-adhesion mesh can be used in the pre-peritoneal space.

While the structures in the above-referenced patent publication prove effective, present principles understand that the ventral wall (mesh) layer can shrink over time owing to tissue ingrowth while the anti-adhesion (peritoneal space) layer does not, which can lead to bunching of the implant. Additionally, present principles recognize that even better implant compliance to reduce patient discomfort may be provided.

Present principles also address facilitating the centering a large implant, which is advanced through a laparoscopic trocar, relative to the hernia defect. This is challenging because the mesh must be rolled in a thick cigar-like fashion to advance it through a narrow cannula in a trocar, unrolled, and then properly positioned centrally over the hernia.

### SUMMARY OF THE INVENTION

Among other advantages, the decrease of mesh mass achieved through the implant design highlighted herewith proves helpful in delivering the implant through a trocar cannula.

Accordingly, in one embodiment a hernia repair implant includes a first layer made of mesh for facing a body structure having a hernia defect to cover the defect while promoting tissue growth into the first layer from the body structure. The implant also includes a second layer opposed to the first layer that is made of anti-adhesion material to prevent growth of tissue into the second layer from body structures contacting the second layer, the second layer being understood to extend radially beyond the first layer. In addition, the implant has at least a first elongated centering strap connected to the first layer that is connected to the first layer at a first radial location. Also, the implant includes at least a first elongated fixation strap connected to the first layer that is connected to the first layer at a second radial location that is more distanced from a center of the first layer than the first radial location.

If desired, the mesh of the implant may define a pore size. The first layer may either be a continuous mesh layer in that it has no openings larger than the pore size, or it may be a skeleton mesh layer defining a periphery and defining at least one opening within the periphery larger than the pore size. Note that the skeleton portion can be interrupted entirely such that islands of mesh can be backed onto the anti-adhesion layer.

Furthermore, in some embodiments the implant also includes a spacer structure between the first and second layers such that the spacer structure distances the first and second layers. The spacer structure may include at least one rounded nodule and/or one sphere. Or, it may establish a spiral shape, or it may include one or more hollow elements each defining a complete enclosure. Yet again, the spacer structure may include plural popcorn elements, it may be petal-shaped with stems of petals being juxtaposed adjacent to each other and ends of petals being radially distant from each other, and/or the structure may be established by any combination of the foregoing structures.

In another aspect, a hernia repair implant includes a first layer made of mesh for facing a body structure having a hernia defect to cover the defect while promoting tissue growth into the first layer from the body structure. The implant also includes a second layer opposed to the first layer and made of anti-adhesion material to prevent growth of tissue into the second layer from body structures contacting the second layer. Additionally, the implant has a structure that is not a flat continuous plane interposed between the first and second layers to distance the layers from each other, rendering the combined structure dynamic and compressible to stimulate better tissue ingrowth via cyclical physiologic loading.

In still another aspect, a method includes advancing, through a trocar, an implant into a patient through an incision adjacent to a portion of a muscle wall to be repaired. The implant includes centering straps connected to a mesh and fixation straps connected to the mesh outboard of where the centering straps are connected. The method also includes advancing the centering straps through the muscle wall to partially deploy the mesh in a centered positioned relative to a defect in the muscle wall. The method then includes advancing the fixation straps through the muscle wall to complete the fixation of the mesh to the muscle wall. With the centering straps, no sutures or other tacking structure is used to center the mesh over the defect but only the centering straps, which also fix the mesh to the wall, are used to center the mesh. This advantageously eliminates a separate suturing step and furthermore permits improved manipulation when centering the mesh compared to suturing a central part of the mesh on or near the defect.

In another aspect, a hernia repair implant has a first layer made of mesh for facing a body structure having a hernia defect to cover the defect while promoting tissue growth into the first layer from the body structure. A second layer is opposed to the first layer and is made of anti-adhesion material to prevent growth of tissue into the second layer from body structures contacting the second layer. The mesh defines a pore size and the first layer is a skeleton mesh layer defining a periphery and defining at least one opening within the periphery larger than the pore size.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1A is a cross-sectional view of a ventral portion of an anterior abdominal wall;

FIG. 1B is a cross-sectional view of FIG. 1A showing a herniation in the ventral wall;

FIGS. 2-5 are schematic diagrams illustrating the implantation of a mesh with centering straps;

FIG. 6 is a plan view of an example mesh shown in FIGS. 2-5 suitably configured for ventral wall hernia repair, showing four centering straps and seven fixation straps;

FIG. 7 is a plan view of an alternate mesh configured for inguinal canal hernia repair;

FIGS. 8-11 are plan views of various embodiments of a skeleton mesh that is flush against an anti-adhesive layer, with the anti-adhesive layer extending radially beyond the skeleton mesh to ensure that tissue in the peritoneal space does not grow around the edge of the anti-adhesive layer into the mesh;

FIG. 12 is a top perspective view of a skeleton mesh along the lines of those shown in FIGS. 8-11 with centering and fixation straps along the lines of the embodiment shown in FIG. 6;

FIG. 13 is a schematic side view illustrating an implant made of a relatively compressible structure interposed between a first mesh layer and an anti-adhesion side layer including a second mesh positioned against the compressible structure and an anti-adhesion sheet, it being understood that in some embodiments the anti-adhesion sheet can be omitted and the second mesh made of anti-adhesion material;

FIG. 14 is a top plan view of an example embodiment of an implant mesh similar to the one shown in FIG. 13 with a spiral-shaped relatively compressible structure appearing through a first mesh layer;

FIG. 15 is a perspective view of the implant shown in FIG. 14;

FIGS. 16-18 are top perspective views of implants with alternate interior relatively compressible structures, with one mesh layer folded away from the other mesh layer to better show the configuration of the relatively compressible structures;

FIGS. 19 and 20 are elevational and perspective views, respectively, of an “island” type skeleton mesh with FIG. 20 omitting the centering straps for clarity; and

FIG. 21 is a perspective view of a strap retrieval tool.

### DETAILED DESCRIPTION OF THE PREFERRED EMBODIMENT

Initially, it is to be understood that although the repair of ventral hernias is particularly referenced herein, the apparatus and methods described herein may be used for other surgical or laparoscopic procedures such as, but not limited to, other instances where a tissue structure of the human body requires strengthening or supporting. Furthermore, although shown in the ventral portion of the abdominal wall and although so described for treatment of ventral hernias, the apparatus and methods described herein may be used for inguinal hernias, pelvic support, and other procedures and areas of the body.

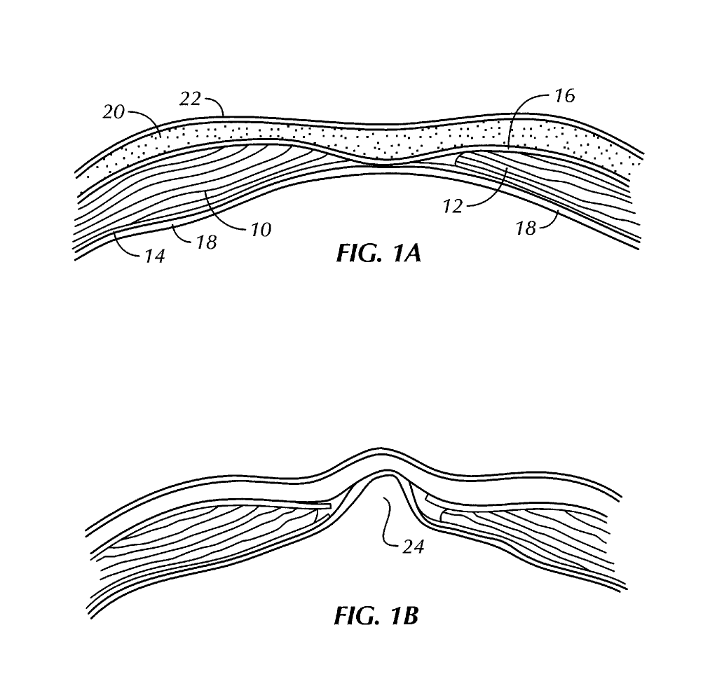

Now initially referring to FIG. 1A, a cross-sectional view of a normal, anterior abdominal wall of the ventral region of the body is shown. As shown, the abdominal wall includes left and right rectus muscles 10 and 12 enclosed and held in place by posterior layers of fascia 14 and anterior layers of fascia 16. These layers of fascia, which are thin, strong fibrous tissue, merge together in the region intermediate the rectus muscles 10 and 12. A thin layer 18, called the peritoneum, covers the posterior side of the posterior fascia 12. The peritoneum 18 is a soft, pliable layer of tissue material and provides an enclosure for the intestines and other internal viscera. A layer of skin composed of the sub dermis 20 and dermis 22 covers the exterior of the anterior fascia 16. FIG. 1B illustrates a condition where a hernia has formed in the wall of the abdomen. The hernial opening is shown at 24. In this example, the hernia is formed by the rupture of the fascia layers 14 and 16 in the region intermediate the rectus muscles 10 and 12. Note that a visceral protrusion can occur not only in the midline but also in the lateral aspect of the abdominal wall. In this case the viscera protrude across the lateral wall musculature being composed by the external and internal oblique and the transverse muscles. In any case, the rupture permits the internal viscera to push the peritoneum 18 in an outward direction, creating a bulge 24 in the skin layers 20 and 22. If not treated, the condition will only worsen with time, with the peritoneal bulge becoming larger.

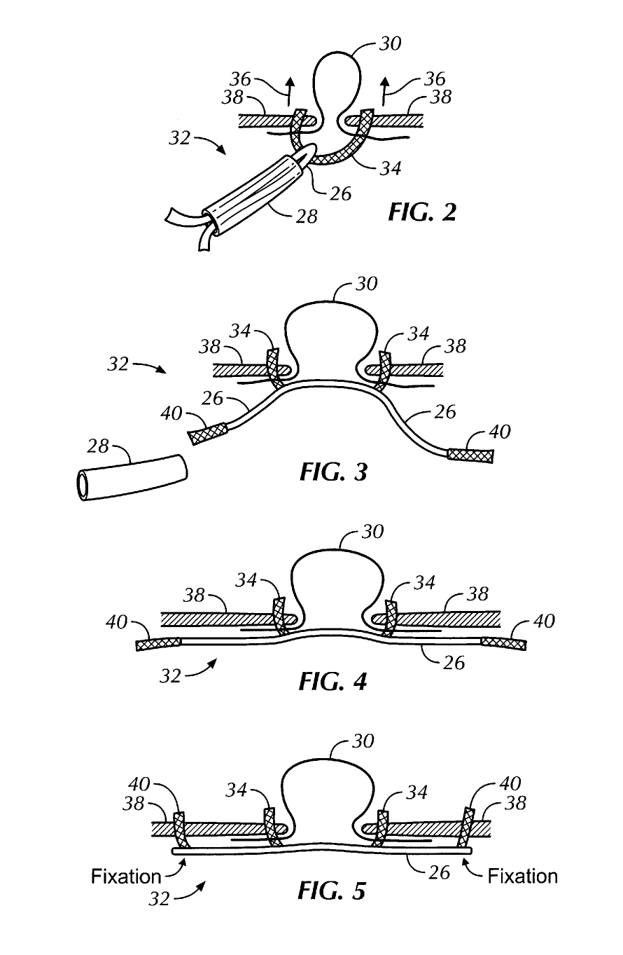

Now referring to FIGS. 2-5, schematic diagrams illustrating the implantation of a mesh with centering straps are shown. It is to be understood that the figures below generally show methods steps in conjunction with devices disclosed herein. Specifically, FIGS. 2-5 show advancing, using a suitable medical instrument, an implant into a patient through an incision adjacent to a portion of a muscle wall to be repaired. As shown and described below, the implant includes centering straps connected to a mesh and fixation straps connected to the mesh outboard of where the centering straps are connected. In other words, the centering straps are connected to the mesh closer to the geometric center of the mesh than are the fixation straps. The centering straps are thus advanced through the muscle wall to partially deploy the mesh in a centered positioned relative to a defect in the muscle wall, and the fixation straps are then advanced through the muscle wall to complete the fixation of the mesh to the muscle wall.

With the centering straps, no sutures or other tacking structure is used to center the mesh over the defect but only the centering straps, which also fix the mesh to the wall, are used to center the mesh. This advantageously eliminates a separate suturing step and furthermore permits improved manipulation when centering the mesh compared to suturing a central part of the mesh on or near the defect since the centering straps permit the surgeon to move the mesh laterally as needed to center the mesh by cinching the straps as necessary to center the mesh.

Furthermore, note that the meshes described herein, including skeleton mesh portions of the implants described herein and the mesh straps described herein, may be constructed of a solid or a permeable material such that they are receptive to tissue ingrowth. Suitable materials for making the meshes may include, but are not limited to, the following: polypropylene mesh such as that distributed by C. R. Bard, Inc. of Murray Hill, N.J. under the trade name “Marlex”; a polyethylene mesh material of the type distributed by E. I. Du Pont de Nemours and Company of Wilmington, Del. under the trade name “Alathon”; a Dacron mesh material or a Nylon mesh material of the type distributed by E.I. Du Pont de Nemours and Company of Wilmington, Del.; Teflon; and silicone.

Additionally, the meshes described herein may be constructed from a metallic mesh or a polymer mesh having interwoven metallic filaments, if desired. These filaments may provide additional strength to the meshes or make the meshes radiopaque for later visualization. The meshes may be a single layer or have a multilayer construction. The meshes may have one or more layers constructed from a bioabsorbable material such that the meshes may be reabsorbed by the body over time.

Now particularly with respect to FIG. 2, it may be appreciated that an implant 26 has been advanced into a patient through, e.g., an incision next to a hernia 30 to be repaired using a suitable medical device 28 (such as, e.g., a trocar and/or protective sheath), it being understood that the implant 26 as shown in FIG. 2 is compressed (e.g., rolled in a cigar-style fashion) to allow advantageous advancement using the device 28. The implant can thus be advanced into the patient using, e.g., laparoscopic techniques and toward the hernia 30 in the ventral wall via the abdominal cavity 32. The hernia 30 has characteristics related to/similar to the hernial opening 24 described above. It may be appreciated from FIG. 2 that the implant 26 can include plural centering straps 34.

If desired, the centering straps 34 may be advanced into the patient first, with the remaining portions of the implant delivered via, e.g., the trocar and sheath, after the straps 34 have been at least partially advanced into the patient having the hernia 30. Advancing the straps 34 first may make advancement of the straps 34 into the abdominal wall 38 less complicated since, e.g., the remaining portions of the implant 26 are less likely to get in the way and obscure a surgeon's view while performing a procedure in accordance with present principles and anchoring the centering straps 34 to place the implant 26 at a desired orientation.

As may be appreciated from the upward arrows 36 shown in FIG. 2, the centering straps 34 are advanced at least partially into the abdominal wall 38 and preferably the centering straps 36 are advanced completely through the abdominal wall such that they are advanced outwardly through the skin of the patient having the hernia 30, including being advanced through the sub dermis and dermis. The above-incorporated parent patent application of which this is a continuation in part discloses various techniques for doing this. Accordingly, it may be appreciated from FIG. 3 that the centering straps 34 are at least partially disposed in the abdominal wall 38 and, owing to being advanced into the abdominal wall 38 at a location radially distant from the hernia 30 itself, the straps 34 at least partially ensure that no excess mesh or another portion of the implant 26 migrates up into the hernia 30. Furthermore, the straps 34, when advanced into the abdominal wall 38, prevent the implant 26 from sagging when, e.g., pneumoperitoneum is released and thus it at least partially eliminates the chances of hernia recurrence and the potential for seroma. As may also be appreciated from FIG. 3, the device 28 is withdrawn from the area of the hernia 30, allowing the implant 26 to begin to expand, unfold, deploy, and/or otherwise assume an intended shape to cover the defect in the abdominal wall 38 caused by the hernia 30 and facilitate tissue growth in accordance with present principles.

Given that FIG. 3 shows the implant 26 being fully removed from the device 28, it may be appreciated that plural fixation straps 40 are also evident on the implant 26. The fixation straps 40 will be described further in reference to FIG. 5. But first, note that as shown in FIG. 4, the implant 26 is shown at least partially covering/blocking/obscuring the hernia 30 in the abdominal wall 38, it being understood that the implant 26 shown in the configuration of FIG. 4 has at least partially assumed its intended shape.

Now in reference to FIG. 5, it may be appreciated that the fixation straps 40 have now been advanced at least partially into the abdominal wall 38. If desired, the fixation, straps 40 may be advanced completely through the abdominal wall 38 such that they are advanced through the skin of the patient with the hernia 30. It may be further appreciated from FIG. 5 that a parietal surface of the implant 26 is now disposed against the abdominal wall 38 to fully cover the hernia 30, thereby facilitating tissue growth in accordance with the principles set forth herein, while also advantageously blocking passage of objects, fluid, organs, tissue, etc. from passing through the hernia 30 at least partially due to the visceral surface of the implant 26 (which may have anti-adhesion characteristics as set forth herein).

Note that either or both of the centering straps 34 and fixation straps 40 may be secured into abdominal wall 38 by way of friction between the straps 34 and 40 and the wall 38 to minimize patient discomfort while still ensuring that the implant 26 remains in its intended position/orientation, and also does not migrate within the abdominal cavity 32. This provides a relatively tension-free anchoring means while also obviating the need to use other tacking methods that may otherwise provide potential points of adhesion and/or tension during the healing process of the patient, which is undesirable due to, e.g., patient discomfort. Eliminating sutures or other tacking devices also enables the implant to move with expansion or contraction of the surrounding tissue as part of the healing process due to tissue changes over time as the wall 38 heals and as incorporation tissue invades the implant 26. In essence, securing the implant using only strap friction better accommodates tissue movement and/or expansion. However, if deemed necessary additional forms of fixation may nonetheless be used, such as, but not limited to, tacking, sutures, fasteners, and clamps.

Notwithstanding the foregoing, it may be appreciated that using only the friction means of abdominal wall attachment provides a relatively tension-free condition in which the implant 26 is secured into its position with sufficient slack so that as surrounding tissue expands or moves, the implant slack helps avoid pulling and possible tearing of surrounding tissue that may otherwise result from an implant that is secured too tightly or does not have any residual slack due to, e.g., tacking or clamps. Accordingly, it may be appreciated that by virtue of the friction created between the abdominal wall 38 and straps 34 and 40, the straps 34 and 40 secure and stabilize the implant 26 while also permitting a desired level of movement the straps 34 and 40 relative to surrounding tissues over time. The relatively tension-free straps 34 and 40, as well as the configuration of the implant 26 that completely covers the hernia 30, provides for substantial slack allowing for long-term natural abdominal wall remodeling which present principles recognize as being particularly important to reducing and fixing hernias. It is to be understood that this type of tension free and fixation free implant may promote better healing, reduce premature tear-out or dislodgement or dislocation and provide increased comfort and acceptance by the patient.

Still addressing the straps 34 and 40, note that while FIGS. 2-5 show that the straps 34 and 40 are shown attached to the implant 26 when advanced into the patient having the hernia 30, in other embodiments the implant 26 may be advanced into the abdominal cavity 32 with the straps 34 and 40 unattached thereto. Thus, the straps 34 and 40 may be advanced at least partially into the abdominal wall 38 while unattached from the implant 26 and then subsequently be coupled/attached to the implant 26. Alternatively or in any desired combination, the implant 26 may be advanced into the abdominal cavity 32 with the straps 34 and 40 unattached, and then subsequently the straps may be attached to the implant 26 prior to the straps 34 and 40 being advanced into the abdominal wall 38. It may be appreciated that advantages of advancing the implant 26 into the abdominal cavity 32 with the straps 34 and 40 unattached may be desired for reasons such as, but not limited to, ease of advancement of the implant 26 into the patient (e.g., if the implant is relatively large and difficult to place into or maneuver using the device 28) and ease of placement of the implant 26 against the abdominal wall 28 to thereby cover the hernia 30.

Continuing in reference to the straps 34 and 40, the straps may be made of a mesh such as a polypropylene mesh that facilitates tissue growth in accordance with present principles. The straps 34 and 40 may be made of any other suitable synthetic materials, biological materials, or combination of materials, if desired. Regardless, it is to be understood that to further facilitate advancement of the straps 34 and 40 at least partially into the abdominal wall 38, the straps 34 and 40 may include surgical needles (not shown in FIGS. 2-5) engaged with respective ends of the straps to facilitate advancement of the straps 34 and 40 into the abdominal wall 38. In some embodiments, the needles are removably engaged with the straps 34 and 40 such that the needles may be disengaged with straps 34 and 40 after the straps 34 and 40 have been at least partially advanced into the abdominal wall.

Also note that in some embodiments, the straps 34 and 40 may be tapered at the ends to be advanced into the abdominal wall. This may facilitate advancement of the straps 34 and 40 through various tissue structures. Accordingly, the reduced lateral profile may reduce friction and the resultant force required to, e.g., pull or push the straps 34 and 40 into the abdominal wail 38. Note that the straps 34 and 40 may be made out of polyethylene, polypropylene, Teflon, nylon, silicone or other suitable polymer in accordance with present principles that may be useful to reduce friction as the straps 34 and 40 pass through tissue in the abdominal wall 38.

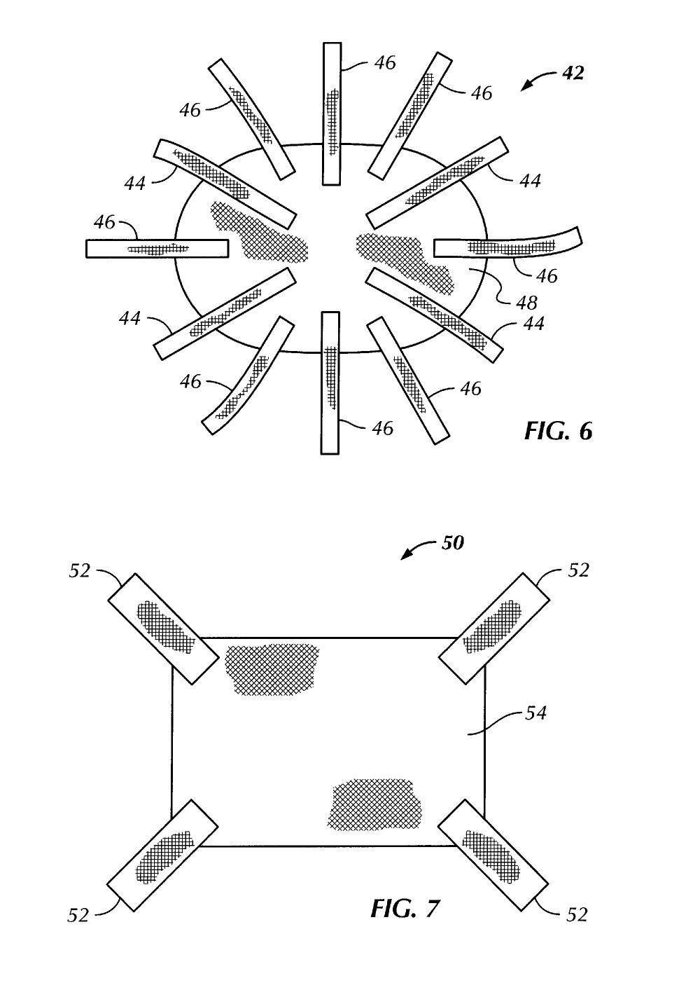

Now addressing FIG. 6, a plan view of an example mesh implant such as the one shown in FIGS. 2-5 suitably configured for ventral wall hernia repair is shown. FIG. 6 shows, for non-limiting illustration, four centering straps 44 and seven fixation straps 46 attached to the mesh implant 42. It is to be understood that the centering straps 44 may be substantially similar in function and configuration to the centering straps 34 described above, while the fixation straps 46 in FIG. 6 may be substantially similar in function and configuration to the fixation straps 40 described earlier. Note that while FIG. 6 shows four straps 44 and seven straps 46, more or fewer straps may be used as desired.

Further, it may be appreciated from FIG. 6 that the body 48 of the implant 42 may be generally circular/radial in shape, though any desired shape may be used to sufficiently cover a hernial opening. Still, it is noted that in FIG. 6 which shows the generally circular/radial implant 42, the centering straps 44 are attached to the implant 42 at radial locations that are less distanced from the center of the implant than where the fixation straps 46 are attached to the implant 42.

Moving on, FIG. 7 shows a plan view of an alternate mesh implant configured for hernia repair. The body 54 of the implant 50 shown in FIG. 7 is generally rectangular in shape, again noting that in other embodiments the implants described herein may be in any suitable geometric or non-geometric shape (e.g., a shape specifically tailored and/or formed by a physician) for covering a hernia opening. FIG. 7 also shows four fixation straps 52 extending diagonally away from respective corners of the body 54. It is to be understood that the straps 52 may be substantially similar in function and configuration to the straps 40 described above, with some differences being considered given the differing shapes of the bodies of the respective implants to which the straps are attached. Moreover, though not shown in FIG. 7, if desired centering straps may also be included on the body 54 of the implant 50 in accordance with present principles, it being understood that the centering straps, if included, would be attached to the body 54 at areas of the body 54 relatively closer to the center of the body 54 than where the straps 52 are attached. Also note that the straps 54 as well as other centering and fixation straps disclosed herein may be, in examples, two centimeters in width and ten centimeters in length in non-limiting embodiments. In addition, note that the body 54 may be, but is not limited to, the dimensions of fifteen centimeters by fifteen centimeters, as well as other implant bodies disclosed herein. However, note that the size and length of one or more elements included on the implants described herein may vary depending on the dimensions of the hernia to be repaired.

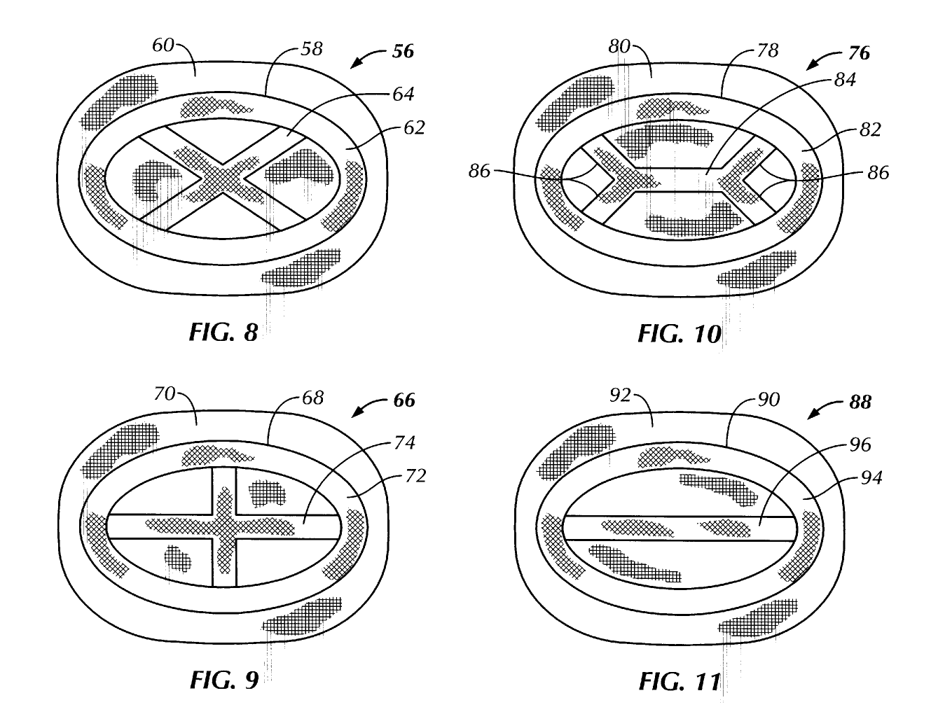

Attention is now made to FIGS. 8-11, which are plan views of various embodiments of a skeleton mesh that is flush against an anti-adhesive layer, with the anti-adhesive layer in some examples extending radially beyond the skeleton mesh (for example, by a few centimeters) to ensure that tissue in the peritoneal space does not contact or become adhered around the edge of the anti-adhesive layer and into the mesh. Advantageously the skeleton mesh has less mass than a continuous mesh, facilitating advancement of the mesh through the trocar. Moreover, because the skeleton mesh offers sufficient yet not excessive room for tissue ingrowth, it is less likely to bunch when tissue grows into it but not into the anti-adhesion layer, as can happen when a completely continuous ingrowth mesh is joined to an anti-adhesion layer.

Additionally, the gaps or islands established by the example skeleton structures described below ensure that scar tissue cannot bridge and thus an undesirable full length contraction of the mesh during healing is avoided. Radial contraction of the mesh caused by such scar tissue growing into the mesh contracts the individual “islands” or mesh portions of the skeleton structure but cannot transmit contraction across the entire length of the anti-adhesion layer. In the embodiments of FIGS. 8-12, a circumferential ring of mesh is shown (which may also be an interrupted ring) inboard of the edge of the anti-adhesion layer. This outer ring provides an ingrowth ring that prevents lifting of the implant and possible entrapment of viscera behind the implant.

Referring first to FIG. 8, an exemplary implant 56 has a skeleton layer 58 made of mesh for facing a body structure having a hernia defect to cover the defect while promoting tissue growth into the skeleton layer from the body structure. The skeleton mesh 58 can be made of any suitable tissue in-growth material such as any of the material described above. The implant 56 also has a second layer 60 that is opposed to the skeleton layer 58. It is to be understood that the second layer 60 is made of anti-adhesion material to prevent growth and/or incorporation of unintended tissue from the abdominal cavity contacting the second layer into the implant 56.

As but one example, the portion of the implant 56 having the skeleton layer 58 may be juxtaposed alongside and/or against an abdominal wall to cover a hernial opening in the abdominal wall such that the second layer 60 faces the abdominal cavity of the patient. Thus, organs such as the patient's bowels will be prevented from sticking to, growing on, being entangled with, etc., the implant 56 by virtue of the anti-adhesion characteristics of the second layer 60 blocking any contact between the organs and the skeleton layer 58.

It may therefore be appreciated that the anti-adhesion elements and materials described herein prohibit ingrowth or attachment of tissue to portions of the implant having the anti-adhesion elements and/or properties. In addition to the second layer 60 having anti-adhesion characteristics, note that in lieu of or in addition to the implant having a second layer such as the layer 60 with anti-adhesion characteristics, other portions of the implants described herein may be coated with an anti-adhesional coating as desired (e.g., on a side to facing away from the abdominal wall and toward the abdominal cavity) to thereby inhibit tissue attachment. Put another way, it may be appreciated that the anti-adhesional characteristics may be particularly useful for those implant surfaces that are exposed to the internal viscera of the abdominal cavity. One example of an adhesion resistant material is a thread of polytetrafluoroethylene polymer material of the type sold under the trade name “Gore-Tex” by W. L. Gore & Associates, Inc. Other non-limiting examples include single sheet polypropylene such as Dipromed, PVDA films, silicone barriers, or biologic or biomimetic meshes.

With reference still being made to FIG. 8, it may be appreciated that the example mesh skeleton layer 58 has a generally oval portion 62 and an “X” patterned portion 64 inside the oval portion 62 extending diagonally relative to the major and minor axes of the oval portion 62 to respective inside edges of the oval portion 62. It may be further appreciated from FIG. 8 (as well as from the respective implant structures of FIGS. 9-12) that the second (anti-adhesion) layer 60 extends radially beyond the skeleton layer 58. Although the “X” portion 64 includes two continuous strips crossing each other, the strips need not be continuous, and instead “islands” of mesh that are not connected to each other can establish the skeleton, tissue ingrowth layer.

FIG. 9 shows an alternate skeletal structure for a layer of an implant to be positioned against the abdominal wall of a patient in accordance with present principles. The implant 66 shown in FIG. 9 has a skeleton layer 68 made of mesh for facing a body structure having a hernia defect. The implant 66 also has a second layer 70 that is opposed to the skeleton layer 68. It is to be understood that the second layer 70 is made of anti-adhesion material to prevent growth of tissue in accordance with present principles.

Note that FIG. 9 shows the mesh skeleton layer 68 being comprised of a generally oval portion 72 and cross-pattern or plus-sign-pattern portion 74 inside the oval portion 72 with mesh extending in vertical and horizontal directions relative to the major and minor axes of the oval portion 72 to respective inside edges of the oval portion 72. Also note that the second layer 70 extends radially beyond the skeleton layer 68.

FIG. 10 shows another alternate skeletal structure for a layer of an implant to be positioned against the abdominal wall of a patient in accordance with present principles. The implant 76 shown in FIG. 10 has a skeleton layer 78 made of mesh for facing a body structure having a hernia defect. The implant 76 also has a second layer 80 that is opposed to the skeleton layer 78. It is to be understood that the second layer 80 is made of anti-adhesion material to prevent growth of tissue in accordance with present principles.

Note that FIG. 10 shows the skeleton layer 78 made of a mesh with a generally oval portion 82 and a pattern inside the oval portion 82 having a central horizontal portion 84 and four diagonal portions 86 extending diagonally away from the two respective ends of the central horizontal portion 84. Note that the description of the internal pattern of the skeleton layer 78 is made relative to the major and minor axes of the oval portion 82 of the skeleton layer 78. Also note that the second layer 80 extends radially beyond the skeleton layer 78.

FIG. 11 shows yet another alternate skeletal structure for a layer of an implant to be positioned against the abdominal wall of a patient in accordance with present principles. The implant 88 shown in FIG. 11 has a skeleton layer 90 made of mesh for facing a body structure having a hernia defect. The implant 88 also has a second layer 92 that is opposed to the skeleton layer 90. It is to be understood that the second layer 92 is made of anti-adhesion material to prevent growth of tissue in accordance with present principles.

Note that FIG. 11 shows the skeleton layer 90 with a generally oval portion 94 and mesh horizontal portion 96 inside the oval portion 94 that extends along the major axis of the oval portion 94 and terminates at inside edges of the oval portion 94. Also note that the second layer 92 extends radially beyond the skeleton layer 90.

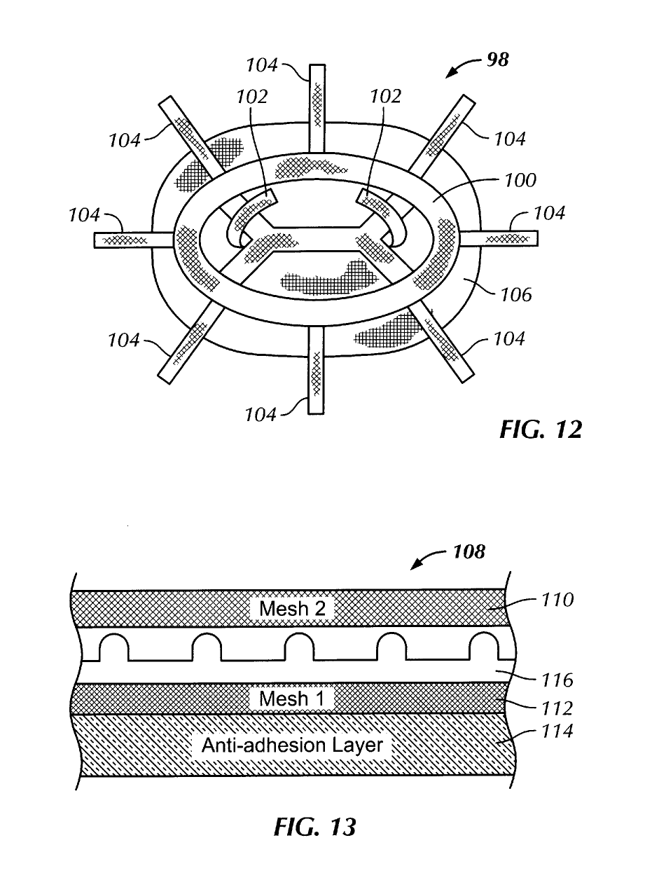

Reference is now made to FIG. 12. FIG. 12 shows a top perspective view of a hernia implant 98 with a skeleton mesh 100 along the lines of those shown in FIGS. 8-11 with elongated centering straps 102 and elongated fixation straps 104 along the lines of the embodiment shown in FIG. 6. FIG. 12 thus shows the skeleton mesh 100 with a second, anti-adhesion layer 106 extending radially beyond the skeleton mesh 100. It may be appreciated that the skeleton mesh 100 is substantially similar in configuration to the skeleton layer 78 described in reference to FIG. 10, though it is to be understood that any of the other skeleton layer configurations described herein may be used in accordance with present principles.

Still in reference to FIG. 12, it is to be appreciated that the elongated centering straps 102 are connected to the skeleton mesh 100. In some embodiments the straps 102 may extend radially beyond the periphery of the skeleton mesh 100, but in other embodiments the straps 102 need not necessarily extend radially beyond the periphery of the skeleton mesh 100 so long as they are long enough to be advanced at least partially into the abdominal wall of a patient as desired. Regardless, note that the centering straps 102 are connected to the skeleton mesh 100 at a first radial location of the skeleton mesh 100 relative to the center of the skeleton mesh 100, a second location to be described shortly.

It may also be appreciated from FIG. 12 that the elongated fixation straps 104 are connected to the skeleton mesh 100 inboard of the anti-adhesion layer edges. Because the straps are passed through the abdominal wall directly in line with their attachment point to the skeletal mesh, the overlap of the anti-adhesion layer prevents contact of viscera to exposed strap material. In some embodiments the straps 104 may extend radially beyond a periphery of the skeleton mesh 100, but in other embodiments the straps 104 need not necessarily extend radially beyond the periphery of the skeleton mesh 100 but are nonetheless long enough to be advanced at least partially into the abdominal wall of a patient as desired. Regardless, note that the fixation straps 104 are connected to the skeleton mesh 100 at a second radial location of the skeleton mesh 100 that is more distanced from a center of the skeleton mesh 100 than the first radial location described in the paragraph above.

Referring specifically to the skeleton mesh 100, note that the skeleton mesh 100 defines a pore size and at least one opening within the periphery that is larger than the pore size (e.g., as may be appreciated from the skeleton configurations of FIGS. 8-11). Furthermore, the skeleton mesh 100 along the lines of the skeleton layers of FIGS. 8-11 is understood to mesh be made of polypropylene in exemplary embodiments, but may be made from other suitable synthetic materials, a biological materials, or combination of materials such as those described herein.

Moving on, reference is now made to FIG. 13, which is a schematic side view illustrating an implant 108 made of a compressible, preferably plastic structure 116 interposed between a first mesh layer 110 and a second mesh layer 112 flush against the second mesh layer 112. An anti-adhesion side layer 114 in accordance with present principles is also shown. However, it is to be understood that in some embodiments the anti-adhesion sheet 114 can be omitted and the second mesh 112 can be made of and/or at least partially coated with an anti-adhesion material.

Furthermore, it may be appreciated from FIG. 13 that the structure 116 is not a flat continuous plane interposed between the first mesh 110 and second mesh 112, and may in some embodiments act as a force/shock absorber providing resilience around the implant 108 and hernial area. Thus, the structure 116 distances the mesh 110 and mesh 112 from each other to facilitate tissue ingrowth into the implant. In the exemplary embodiment shown in FIG. 13, the structure 116 includes plural rounded nodules with vacant spaces in between nodules and with flat portions opposite the rounded ends of the nodules to thereby structurally connect the nodules, in other words, a corrugated-like structure. It is to be understood that in other embodiments other configurations may be used, such as plural spheres at least comprising the compressible structure to be interposed between the two meshes 110 and 112. Ribs made from mesh may also be used.

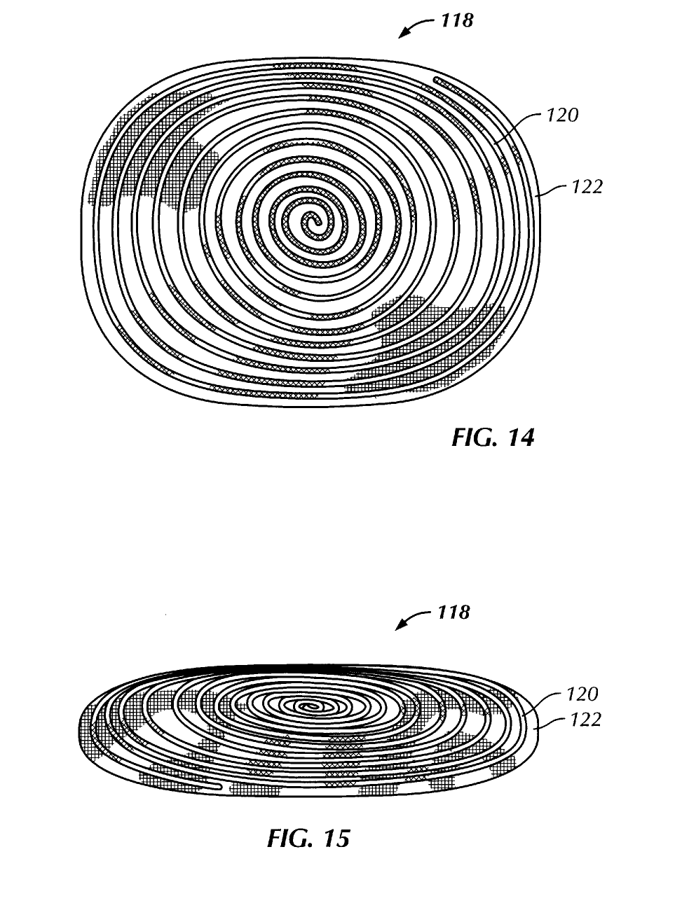

Turning now to FIG. 14, a top plan view of an example embodiment of an implant mesh 118 similar to the one shown in FIG. 13 except with a spiral-shaped compressible structure 120 (rather than rounded nodules) appearing through a first mesh layer 122 in accordance with present principles is shown. The spiral shape of the structure 120 may also be appreciated from the perspective view of the implant 118 shown in FIG. 15.

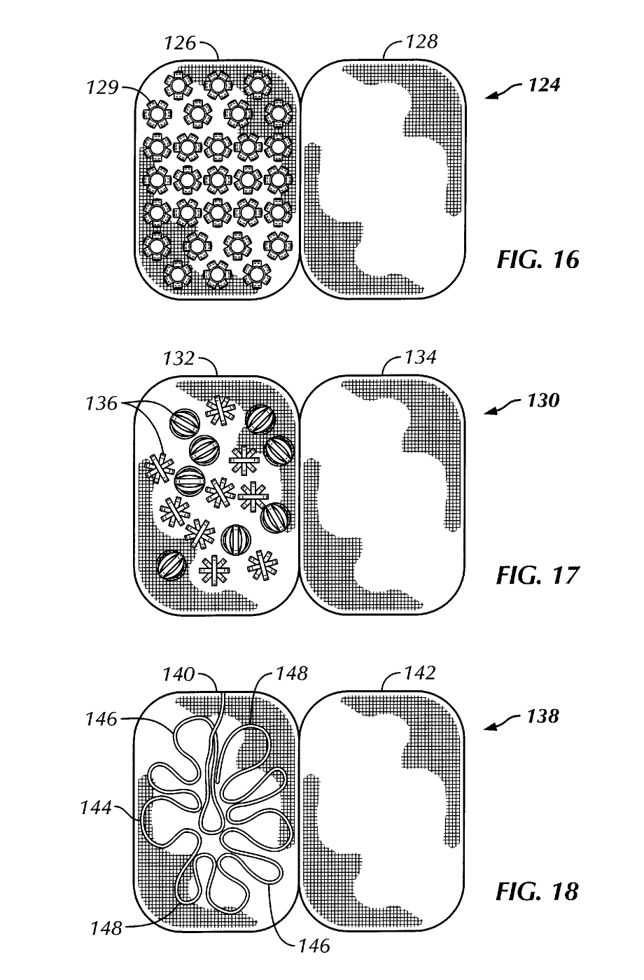

Reference is now made to FIGS. 16-18, which are top perspective views of implants in accordance with present principles having alternate compressible structures. Note that FIGS. 16-18 show one mesh layer folded away from the other mesh layer to better show the configuration of the respective structures of the figures.

Describing FIG. 16, it may be appreciated that an implant 124 includes a first mesh layer 126 and a second mesh layer 128. FIG. 16 also shows a structure 129, which includes plural hollow elements each defining a complete enclosure. If desired, the hollow elements of the structure 129 may be ring-like and/or cylindrical with an inner generally circular core supporting at least one generally circular mesh wing extending outward therefrom.

Describing FIG. 17, it may be appreciated that an implant 130 includes a first mesh layer 132 and a second mesh layer 134. FIG. 17 also shows a compressible structure 136, which is comprised of plural popcorn elements, which may have differing “popcorn” configurations as shown. For example, the popcorn configurations may essentially resemble abstract origami shapes, may resemble the shapes and variances of popcorn, may be comprised of various overlapping circular strips to comprise ball-like shapes, etc.

Describing FIG. 18, it may be appreciated that an implant 138 includes a first mesh layer 140 and a second mesh layer 142. FIG. 18 also shows a compressible structure 144, which is petal-shaped and includes stems of petals 146 that are juxtaposed adjacent to each other, as well as ends of petals 148 that are radially distant from the center of the implant 138.

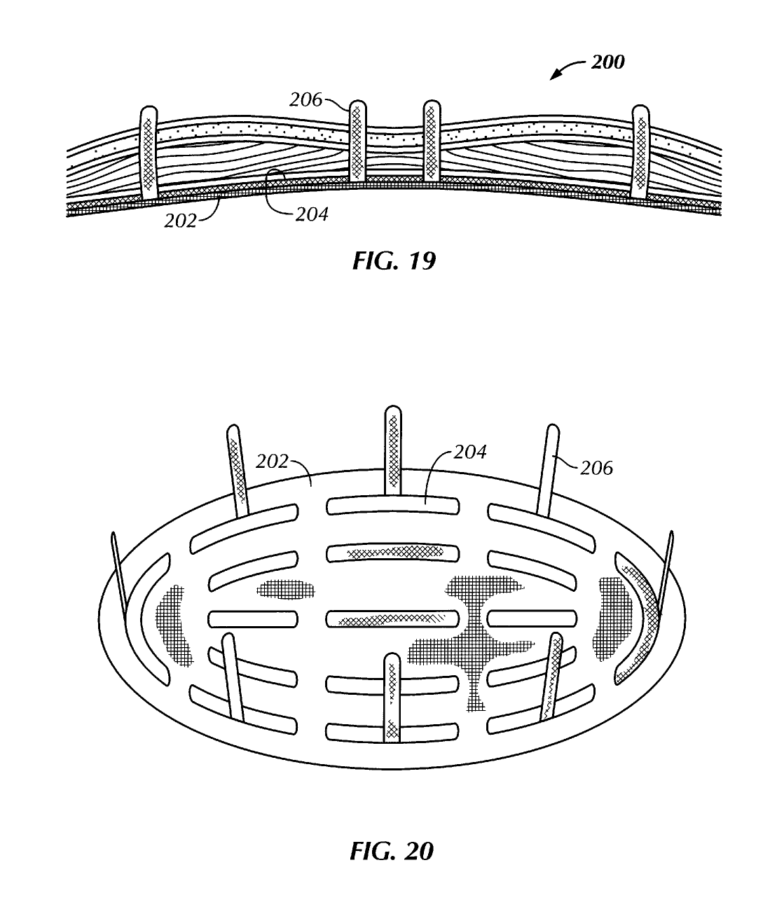

FIGS. 19 and 20 show a skeleton-style mesh 200 including an anti-adhesion layer 202 supporting an island-style skeleton mesh 204 composed of islands of tissue ingrowth mesh that do not touch each other except by being disposed on a common anti-adhesion layer, i.e., at least some of the tissue ingrowth-promoting islands are not connected to another tissue ingrowth-promoting island by tissue ingrowth-promoting structure, although all portions of the mesh 204 may be supported on the anti-adhesion layer 202. Note that the islands in FIG. 20 are formed in an outer interrupted ring and three inner interrupted lines of somewhat elongated islands. Fixation straps 206 rise from islands in the outer ring; centering straps may also be used, connected to some of the inner islands, in accordance with description above.

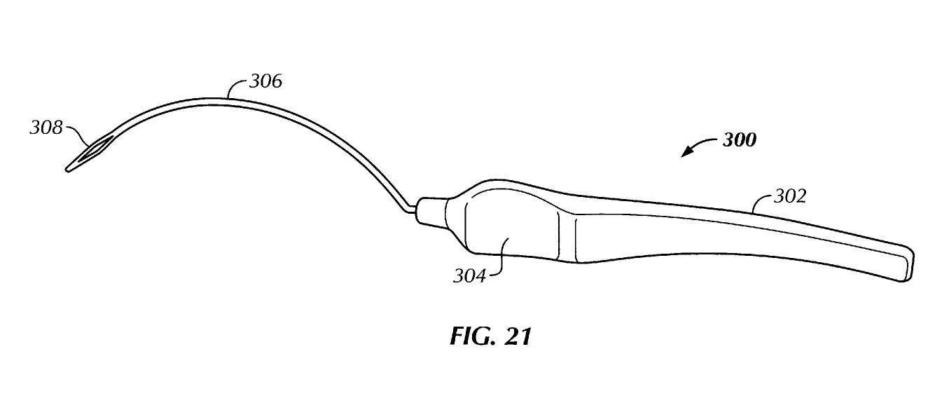

FIG. 21 shows an example strap retrieval tool 300 with an elongated rigid plastic handle 302 and a thumb indent 304 configured for receiving a surgeon's thumb for gripping purposes. An elongated curved almost semi-circular metal retriever 306 extends distally away from the handle 302 as shown, terminating in a slit or eye 308 through which one of the centering or fixation straps discussed above can be passed to thereby engage the retriever 306 with the strap. When open surgery is used the retriever 306 is advanced into the patient through subcutaneous tissue and muscle layers into the peritoneum, whereas in laparoscopic surgery the retriever 306 is advanced into the patient transcutaneously. The surgeon engages the strap with the eye 308 and pulls the tool 300 with strap back through various tissue shown in FIG. 19 to extend outside the patient as shown. The straps can then be trimmed and the tissue tented outwardly so the straps slide back into the tissue, remaining in contact with the tissue for fixation purposes through friction.

It may now be appreciated based on all of the foregoing the implants described herein may be made relatively oversized compared to the size of the hernia. Any such relatively larger implant may improve its adhesion to the abdominal wall. An implant sized larger than the hernia may in some embodiments be 1.5 times larger than the area of the hernia, or may be two times larger than the area of the hernia.

It may also be appreciated that the anti-adhesion portions of the implants described herein may extend radially past the polypropylene mesh elements facilitating tissue growth such that, e.g., organs are not at risk of contacting the mesh elements. Moreover, the implants may be trimmable such that they may be trimmed while in the abdominal cavity once the implant is advanced into the patient but before the implant is placed at a desired location against the abdominal wall. The composition of the implant, at least a portion being made out of, e.g., polypropylene, allows for such trimming. Trimming may be advantageous to shape an implant in accordance with present principles to uniquely conform to and/or uniquely cover a hernia.

While the particular IMPLANT FOR HERNIA REPAIR is herein shown and described in detail, it is to be understood that the subject matter which is encompassed by the present invention is limited only by the claims.

What is claimed is:

1. Hernia repair implant comprising:
a first layer made of mesh for facing a body structure having a hernia defect to cover the defect while promoting tissue growth into the first layer from the body structure;
a second layer opposed to the first layer and made of anti-adhesion material to prevent growth of tissue into the second layer from body structures contacting the second layer; and
at least a first elongated fixation strap connected to the first layer at a first radial location, wherein the second layer extends radially beyond the first layer.

2. The implant of claim 1, comprising at least a first elongated centering strap connected to the first layer at a second radial location that is closer to a center of the first layer than the first radial location at which the fixation strap is connected.

3. The implant of claim 1, wherein the mesh defines a pore size, and the first layer is a continuous mesh layer in that it has no openings larger than the pore size.

4. The implant of claim 1, wherein the mesh defines a pore size and the first layer is a skeleton mesh layer defining a periphery and defining at least one opening within the periphery larger than the pore size.

5. The implant of claim 4, wherein the skeleton mesh layer includes at least two islands of mesh not connected to each other by tissue ingrowth-promoting structure.

6. The implant of claim 1, wherein the implant further comprises at least one spacer structure between the first and second layers such that it distances the first and second layers, wherein the structure includes at least a rounded nodule and/or sphere.

7. The implant of claim 1, wherein the implant further comprises at least one spacer structure between the first and second layers such that it distances the first and second layers, wherein the structure establishes a spiral shape.

8. The implant of claim 1, wherein the implant further comprises at least one spacer structure between the first and second layers such that it distances the first and second layers, wherein the structure includes plural hollow elements each defining a complete enclosure.

9. The implant of claim 1, wherein the implant further comprises at least one spacer structure between the first and second layers such that it distances the first and second layers, wherein the structure includes plural popcorn elements.

10. The implant of claim 1, wherein the implant further comprises at least one spacer structure between the first and second layers such that it distances the first and second layers, wherein the structure is petal-shaped and includes stems of petals that are juxtaposed adjacent to each other and ends of petals that are radially distant.

11. Hernia repair implant comprising:
a first layer made of mesh for facing a body structure having a hernia defect to cover the defect while promoting tissue growth into the first layer from the body structure;
a second layer opposed to the first layer and made of anti-adhesion material to prevent growth of tissue into the second layer from body structures contacting the second layer; and
a structure that is not a flat continuous plane interposed between the first and second layers to distance the layers from each other and facilitate tissue ingrowth into the implant.

12. The implant of claim 11, wherein the mesh defines a pore size, and the first layer is a continuous mesh layer in that it has no openings larger than the pore size.

13. The implant of claim 11, wherein the mesh defines a pore size and the first layer is a skeleton mesh layer defining a periphery and defining at least one opening within the periphery larger than the pore size.

14. The implant of claim 11, wherein the structure distancing the layers from each other includes at least a rounded nodule and/or sphere.

15. The implant of claim 11, wherein the structure distancing the layers from each other establishes a spiral shape.

16. The implant of claim 11, wherein the structure distancing the layers from each other includes plural hollow elements each defining a complete enclosure.

17. The implant of claim 11, wherein the structure distancing the layers from each other includes plural popcorn elements.

18. The implant of claim 11, wherein the structure distancing the layers from each other is petal shaped and includes stems of petals that are juxtaposed adjacent to each other and ends of petals that are radially distant

19. Method, comprising:
advancing, through a trocar, an implant into a patient through an incision adjacent to a portion of a muscle wall to be repaired, the implant including centering straps connected to a mesh and fixation straps connected to the mesh outboard of where the centering straps are connected;
advancing the centering straps through the muscle wall to partially deploy the mesh in a centered positioned relative to a defect in the muscle wall; and
advancing the fixation straps through the muscle wall to complete the fixation of the mesh to the muscle wall, wherein no sutures or other tacking structure is used to center the mesh over the defect but only the centering straps, which also fix the mesh to the wall, are used to center the mesh.

20. The method of claim 19, wherein either or both the centering straps and fixation straps, once advanced through the muscle wall, are secured to the muscle wall by way of friction between the straps and the muscle wall.

21. The method of claim 19, wherein the either or both the centering straps and fixation straps have needles engaged with respective ends of the straps, the needles facilitating advancement of the straps through the muscle wall.

22. The method of claim 21, wherein the needles are removably engageable with the straps such that the needles may be disengaged with straps after the straps have been advanced at least partially through the muscle wall.

23. Hernia repair implant comprising:
a first layer made of mesh for facing a body structure having a hernia defect to cover the defect while promoting tissue growth into the first layer from the body structure; and
a second layer opposed to the first layer and made of anti-adhesion material to prevent growth of tissue into the second layer from body structures contacting the second layer, wherein the mesh defines a pore size and the first layer is a skeleton mesh layer defining a periphery and defining at least one opening within the periphery larger than the pore size.

24. The implant of claim 23, comprising:
at least a first elongated centering strap connected to the first layer and connected to the first layer at a first radial location; and
at least a first elongated fixation strap connected to the first layer and connected to the first layer at a second radial location that is more distanced from a center of the first layer than the first radial location.

25. The implant of claim 23, wherein the second layer extends radially beyond the first layer.
